(19) United States
(12) Patent Application Publication
(10) Pub. No.: US 2006/0173218 A1
(43) Pub. Date: Aug. 3, 2006

(54) Preparation of thiols

(75) Inventors: Christian Muller, Mannheim (DE); Markus Weber, Ludwigshafen (DE); Eckhard Stroefer, Mannheim (DE); Veronika Wloka, Mannheim (DE); Jurgen Faderl, Ilvesheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/300,620

(22) Filed: Dec. 14, 2005

(30) Foreign Application Priority Data
Dec. 15, 2004 (DE) 10 2004 060 320.0

(51) Int. Cl. C07C 319/00

(52) U.S. Cl. 568/61

(57) ABSTRACT

The present invention relates to a process for preparing alkyl thiols by adding hydrogen sulfide to double bonds of C₆-C₂₀ olefins at a temperature of from 20 to 150° C. and a pressure of from 1 to 40 bar, wherein the reaction is carried out in the presence of at least one organic, liquid acid, and to the use of organic, liquid acids as a catalyst for increasing the selectivity and/or reaction rate in the addition of hydrogen sulfide to double bonds of C₆-C₂₀ olefins to prepare alkyl thiols.

The present invention relates to a process for preparing alkyl thiols by adding hydrogen sulfide to the double bonds of olefins.

Alkyl thiols having from 10 to 30 carbon atoms are known compounds. Alkyl thiols or mixtures of these compounds are obtained typically by acid-catalyzed electrophilic addition of hydrogen sulfide (H₂S) to olefins. According to the Markovnikov rule, a tertiary thiol is formed from olefins which bear at least three alkyl substituents on their double bond and a secondary thiol from linear olefins.

Secondary thiols find use as fragrances, as components in lubricant formulations and as hardeners for epoxy resins. In addition, they are used advantageously as intermediates in the preparation of surface-active compounds.

Tertiary thiols are used as molar mass regulators in polymerizations, in particular for free-radical polymerizations of vinylic monomers, for example polymerization of butadiene, styrene, carboxylated styrene, acrylic acid, acrylonitrile, acrylic esters, vinyl ethers or mixtures thereof.

Ullmann's Encyclopedia of Industrial Chemistry, 6^{th} edition, 2000 Electronic Release (Wiley VCH Verlag GmbH, Weinheim, Germany, 2000) gives an overview of known methods for preparing alkyl mercaptans under the heading “Thiols and Organic Sulfides”, point 1.3, “Production of Aliphatic Thiols”, under point 1.3.2 “From Alkenes”. Common olefin mixtures which are reacted with hydrogen sulfide over an acidic catalyst to give tert-dodecyl mercaptan are trimerized isobutene and tetramerized propene. Both are known mixtures of highly branched alkenes which were formerly used to a relatively large extent for preparing anionic surfactants. In the selection of the catalyst, it has to be ensured that the olefin or olefin mixture used does not have too high a polymerization tendency over the selected catalyst, since the catalyst is deactivated if oligomers or polymers accumulate on it, which necessitates more frequent catalyst changes and can thus impair the economic viability of the process.

P. Bapseres, Chim. Ind. (Paris) 90 (1963), p. 358 ff. discloses a process for preparing tert-dodecyl thiol from tetrameric propene and hydrogen sulfide at −40° C. in the presence of boron trifluoride or aluminum trichloride as a catalyst. J. F. Franz and K. I. Glass, Chem. Eng. Prog. 59 (7), 1963, page 68 ff. teach a process for preparing tert-dodecyl thiol from tetrameric propene and hydrogen sulfide at from 49° C. to 71° C. in the presence of a boron trifluoride catalyst.

EP 0122654 discloses a process for preparing secondary thiols having from 10 to 22 carbon atoms at a temperature of from 40 to 140° C. and a pressure of from 10 to 100 bar in the presence of a zeolite catalyst.

GB 625 646 describes a process for hydrogen sulfide addition to trimeric isobutene with a clay catalyst which is activated as desired with sulfuric or phosphoric acid.

U.S. Pat. No. 3,214,386 teaches the use of a mixture of phosphoric acid, boron trifluoride and an alcohol having from 1 to 5 carbon atoms as a catalyst in the addition of hydrogen sulfide to olefins having from 9 to 16 carbon atoms.

In the prior art processes, solid acids such as ion exchange resins and zeolites, or mixtures of phosphoric acid, and boron trifluoride and an alcohol, are used as catalysts in the addition of hydrogen sulfide to double bonds.

Disadvantages of the use of solid compounds as a catalyst are:
Transport processes within the solid (pore) often constitute the rate-determining reaction step. Large reactors having large amounts of catalyst are the consequence.
Ion exchange resins are thermally sensitive and, owing to the exothermic reaction, require constant heat removal from the reactive sites. This has the consequence of expensive parallel design, for example in the form of tube bundle reactors.
Ion exchange resins are mechanically sensitive and can be used without significant attrition only in fixed beds.
Zeolites lose their acidic action very rapidly and have to be regenerated in a costly and inconvenient manner outside the reactor, for example at 500° C.
The zeolite powders consist partly of very small particles in the range of a few micrometers which have to be removed in a very costly and inconvenient manner from the reaction mixture. When the removal takes place outside the reactor, the catalyst also has to be recycled.
Fixed beds composed of shaped bodies based on zeolite powder are worn mechanically while liquids, for example, flow through (for the disadvantage of small particles see below).

### SUMMARY OF THE INVENTION

It is an object of the present invention to provide a process for preparing alkyl thiols by adding hydrogen sulfide to double bonds of olefins, in which the abovementioned disadvantages, which result from the use of a solid, acidic catalyst, can be avoided.

This object is achieved by a process for preparing alkyl thiols by adding hydrogen sulfide to double bonds of C₆-C₂₀ olefins at a temperature of from 20 to 150° C. and a pressure of from 1 to 40 bar, wherein the reaction is carried out in the presence of at least one organic, liquid acid.

### BRIEF SUMMARY OF THE FIGURE

The figure illustrates the progress as measured as a function of the reaction time for example 1 according to the process of die invention.

### DETAILED DESCRIPTION OF THE INVENTION

In the process according to the present invention, C₆-C₂₀ olefins, preferably C₆-C₁₈ olefins, more preferably C₁₀-C₁₆ olefins, most preferably C₁₂ olefins, are used. It is possible to use mixtures of olefins having different carbon number and/or different substitution pattern, or uniform olefins. These olefins or mixtures of olefins may be obtained, for example, by cracking of paraffin wax, oligomerization of ethene or metathesis of hexenes. The resulting products have for the most part a linear structure, while olefins which are obtained by oligomerization of propene and/or butenes are branched.

The olefins which can be used in accordance with the invention may have one or more double bond(s) per molecule. Preference is given to using olefins which have one double bond per molecule, known as monoolefins.

The olefins which can be used in the process according to the invention may either be α-olefins having a terminal double bond or the double bond may also be present internally in the hydrocarbon.

Such linear internal olefins may be obtained, for example, by chlorination-dechlorination of paraffins, by paraffin dehydrogenation and by α-olefin isomerization. As a result of the preparation process, the olefins or olefin mixtures which can be used may comprise impurities, for example aromatic compounds and/or saturated hydrocarbons, in a proportion of up to 3% by weight These impurities do not influence the process according to the invention.

The olefins may be linear or have one or more alkyl substituents along the main carbon chain. When olefins are used in the process according to the invention whose double bond is terminal or has two substituents, secondary thiols are obtained, i.e. the carbon atom bearing the —SH functionality is bonded to two further carbon atoms. When olefins are used which bear at least three substituents on the double bond, tertiary thiols are obtained, i.e. the carbon atom bearing the —SH functionality is bonded to three further carbon atoms. Tertiary alkyl thiols are prepared preferentially by the process according to the invention.

Very particular preference is given to using olefins having 12 carbon atoms in the process according to the invention. As a result of the preparation process, the olefin component used may have contaminations by olefins having a carbon number deviating from 12 up to a proportion of 5% by weight, preferably up to a proportion of 3% by weight.

The dodecenes which can be used in the, process according to tie invention correspond especially preferably to one or more olefins which are derived from the compounds below.

Olefins which are derived from n-dodecane (I) 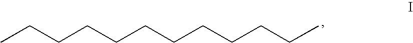
olefins which are derived from 5-methyl-n-undecane (II) 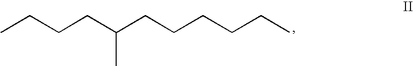
olefins which are derived from 4-ethyl-n-decane (III) 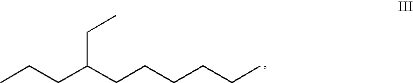
olefins which are derived from 5,6-dimethyl-n-decane (IV) 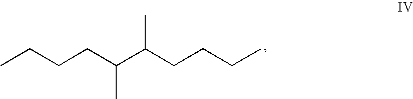
olefins which are derived from 5-ethyl-6-methyl-n-nonane (V), 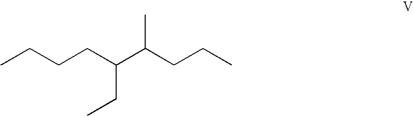
and olefins which are derived from 4,5-diethyl-n-octane (VI)

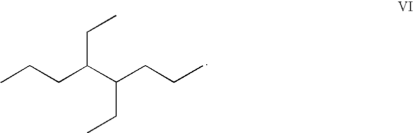

“Derived olefin” refers to an olefin which is formed in a formal sense from the alkane in question by dehydrogenation, i.e. removal of two hydrogen atoms from adjacent carbon atoms to form a double bond between these carbon atoms, the carbon skeleton remaining unchanged. It is neither possible nor necessary to specify the location of the double bond precisely, since the double bond migrates along the carbon chain both in customary methods of preparing such mixtures (for example as specified below) and in the reaction of olefins with hydrogen sulfide. The addition of hydrogen sulfide to olefins in the presence of acidic catalysts is a reversible reaction, although the double bond can form differently to the way in which it was present in the carbon chain. Overall, the position of the double bond in the carbon skeletons and thus also the position of the thiol group, according to the conditions employed, may be under thermodynamic or else kinetic control.

In the process according to the invention, preference is given to using a hydrocarbon mixture which comprises at least 10% by weight, preferably at least 12% by weight and more preferably at least 13% by weight, and at most 18% by weight, preferably at most 16% by weight and more preferably at most 15% by weight, of olefin derived from n-dodecane (I), at least 25% by weight, preferably at least 30% by weight and more preferably at least 34% by weight, and at most 40% by weight, preferably at most 38% by weight and more preferably at most 36% by weight, of olefm derived from 5-methyl-n-undecane (II),

at least 25% by weight preferably at least 30% by weight and more preferably at least 32% by weight, and at most 40% by weight, preferably at most 38% by weight and more preferably at most 34% by weight, of olefim derived from 4-ethyl-n-decane (III),

at least 2% by weight, preferably at least 4% by weight and more preferably at least 5% by weight, and at most 8% by weight, preferably at most 7% by weight, of olefin derived from 5,6dimethyl-n-decane (IV),

at least 5% by weight, preferably at least 6% by weight and more preferably at least 8% by weight, and at most 12% by weight, preferably at most 10% by weight, of olefin derived from 5-ethyl-6-methyl-n-nonane (V),

at least 1% by weight, preferably at least 2% by weight, and at most 5% by weight preferably at most 4% by weight and more preferably at most 3.5% by weight of olefin derived from 4,5-diethyl-n-octane (VI)

and at most 5% by weight, preferably at most 3% by weight, of other hydrocarbons, with the proviso that the sum of the proportions of the components is 100% by weight.

tert-Dodecyl thiol is prepared preferentially from dodecene and hydrogen sulfide by the process according to the invention. In addition to tertiary dodecyl thiols, primary and/or secondary dodecyl thiols may also be present

The configurtion (cis- or trans-configuration isomerism) of the olefins used is not important. In general, the olefms are used in the configuration (or in the form of the mixture of configuration isomers) in which they are obtained, which usually corresponds to the thermodynamically predefined relative stability of the isomers.

The reaction of olefifs (VII) with hydrogen sulfide proceeds generally according to the following reaction scheme

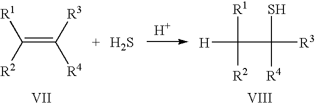

The alkyl thiols (VII) formed during the reaction may react further with a further equivalent of olefin (VII) to give the corresponding thioethers (IX)

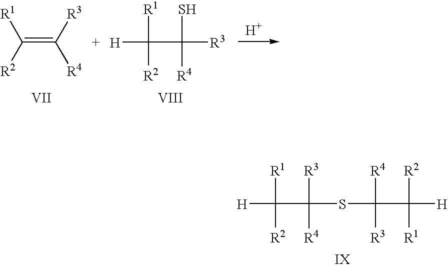

One advantage of the process according to the invention over the prior art processes is that the corresponding alkyl thiols (VIII) are formed selectively from olefins and hydrogen sulfide without the corresponding tioethers (IX) forming in significant amounts.

The process according to the invention is carried out at a temperature of from 20 to 150° C., preferably from 30 to 100° C., more preferably from 35 to 90° C.

The process according to the invention is carried out at a pressure which is above standard pressure. This pressure is generated by injecting hydrogen sulfide, inert gases, for example nitrogen or noble gases, or mixtures of hydrogen sulfide and inert gases. After the gases mentioned have been injected, the process is carried out at a pressure of from 1 to 40 bar, preferably from 5 to 20 bar, more preferably from 7 to 15 bar.

The process according to the invention differs from the processes specified in the prior art in that the reaction of the olefins with hydrogen sulfide is carried out in the presence of at least one organic, liquid acid.

Significant advantages of the use of organic, liquid acids over solid acids are:
Owing to the higher acid strength and acid site density, distinctly more rapid reaction rates are observed, with the consequence of a correspondingly smaller reaction volume. The smaller reaction volume results in a smaller amount of the very poisonous substance hydrogen sulfide in the reactor in comparison to prior art processes.
The heat transfer into and out of the reaction mixture, owing to the miscibility of the liquids involved, for example in mixing pumps and nozzles, presents no problems. In contrast, solids are mechanically sensitive, are worn and are discharged from the reactor in the form of ultrafine solid particles.

The process according to the invention may be carried out in the presence of any organic, liquid acid. Preferred organic liquid acids are aliphatic carboxylic acids having a total of from 1 to 12 carbon atoms. The hydrocarbon radical may be linear or branched, saturated or unsaturated. The carboxylic acids which can be used in the process according to the invention have from one to four, preferably one or two, carboxylic acid functions.

In the process according to the invention, aromatic carboxylic acids having a total of from 7 to 15 carbon atoms may also be used. These have from one to four, preferably one or two, carboxylic acid functions.

In a further particularly preferred embodiment, alkylsulfonic acids of the general formula (X)
R—SO₃H (X)
are used in the process according to the invention, where R is a linear or branched, saturated or unsaturated alkyl radical having from 1 to 10 carbon atoms, preferably a linear or branched, saturated alkyl radical having from 1 to 5 carbon atoms. This alkyl radical may also be mono-, di- or trisubstituted by substituents, for example fluorine atoms. In the process according to the invention, particular preference is given to using an acid selected from the group consisting of methanesulfonic acid, trifluoromethanesulfonic acid, ethanesulfonic acid and propanesulfonic acid. Very particular preference is given to using methanesulfonic acids

When organic, liquid acids are used as catalysts in the addition of hydrogen sulfide to the double bond(s) of olefins, outstanding selectivities and high reaction rates are achieved.

In the process according to the invention, the suitable acids mentioned are preferably used undiluted

Preference is given to not adding any solvents to the reaction mixture. The process is preferably carried out in the absence of a solvent in substance.

The organic, liquid acid is preferably insoluble in the reaction mixture, so that two liquid phases are present.

Preference is given to carrying out the process according to the invention by introducing one or more suitable olefins together with the liquid acid into a reaction vessel and mixing them. Suitable reaction vessels are known to those skilled in the art. Particularly suitable reaction vessels may be reaction mixing pumps which have a rotationally symmetric mixing chamber and a mixing rotor driven in rotation therein. To achieve particularly good mixing of the at least two components, at least one inlet orifice is provided for each component. Such a mixing pump is disclosed, for example, in DE 422 0239.

In the process according to the invention, the molar ratio of acid to olefin (n(acid)/n(olefin)) is preferably from 0.1 to 10, more preferably from 0.5 to 5, most preferably from 0.8 to 3.

The process according to the invention is carried out preferably in a fully inertized reactor. To this end, for example, before the substrates are added, the air-containing gas phase which is present in the reactor is exchanged for an inert gas. This exchange may be effected by repeatedly, for example twice or three times, lowering the pressure (evacuating) in the reaction vessel and injecting the inert gas. The gas exchange may equally be effected by repeatedly, for example twice or three times, injecting an inert gas with subsequent decompression to standard pressure. The inert gas used may, for example, be one selected from the group of nitrogen, helium, neon, argon and mixtures of two or more thereof.

The reaction is carried out until the conversion is generally greater than 70%, preferably greater than 80%, more preferably greater than 85%. The product may be isolated by methods kown to those skilled in the art, for example by phase separation and/or extraction, and purified if appropriate, for example by distillation. If appropriate, acid present in the product solution after the reaction may be neutralized by adding the appropriate amount of one or more bases. Suitable bases are known to those skilled in the art; examples include potassium hydroxide and/or sodium hydroxide in solid or dissolved form.

The process according to the invention may be carried out continuously, semicontinuously or batchwise. In one embodiment, the process according to the invention may be operated continuously, i.e. the product is removed continuously and the substrates are fed continuously according to their consumption, so that constant concentrations of all substances present are present on average in the reaction vessel. Reaction vessels suitable for the continuous procedure are known to those skilled in the art. Examples are tubular reactors, stirred reactors, circulation reactors, preferably in each case with high mixing energy input for mixing the two liquid phases. Particularly suitable reaction vessels may be reaction mixing pumps, as disclosed, for example, in DE 422 0239.

In a further embodiment, the process according to the invention may be carried out semicontinuously, i.e. the substrates are mixed, the reaction is started and products which are formed are removed continuously, for example by distillation.

In the batchwise procedure, the substrates involved are mixed, the reaction is started and, on completion of the reaction, the reaction mixture as a whole is worked up by suitable methods, for example distillation.

The present invention also relates to the use of organic, liquid acids as a catalyst for increasing the selectivity and/or the reaction rate in the addition of hydrogen sulfide to double bonds of C₆-C₂₀ olefins, preferably of dodecene, to prepare alkyl thiols, preferably tert-dodecyl thiol.

Suitable organic acids have already been specified above. Particular preference is given to using alkanesulfonic acids of the general formula (X)
R—SO₃H (X)
where R is a linear or branched, saturated or unsaturated alkyl radical having from 1 to 10 carbon atoms, preferably a linear or branched, saturated alkyl radical having from 1 to 5 carbon atoms, which may be mono-, di- or trisubstituted by substituents, for example fluorine atoms. Particular preference is given to methanesulfonic acid.

The examples which follow illustrate the invention in detail without restricting it.

### EXAMPLES

### Example 1

### Process According to the Invention

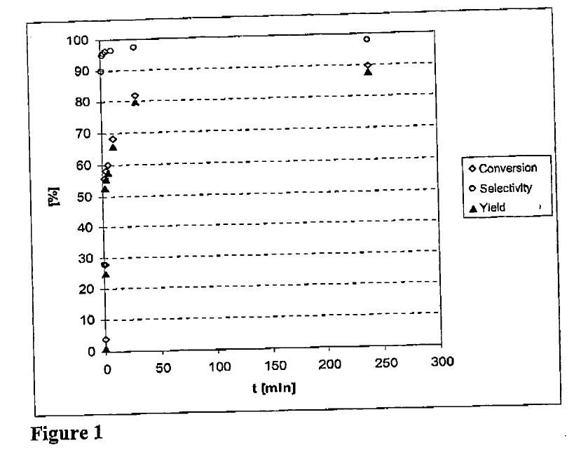

10.6 g of methanesulfonic acid and 40.3 g of dodecene are introduced into the system via a funnel. After the system has been closed, the reaction mixing pump is put into operation (rotation rate: 2800 min⁻¹) and the entire pumped circulation system, which is manufactured in jacketed design including the pump head, is brought to the start temperature of 40° C. The reaction is started by injecting hydrogen sulfide gas to a total pressure of 14 bar. The reaction progress is measured as a function of the reaction time (see FIG. 1). To this end, samples are taken from the system by means of three-way taps. It was ensured that the liquid fill level of the plant does not fall to such an extent tat the reaction mixing pump runs dry, i.e. the amount of liquid above the minimum flu level at the start of the experiment has to at least correspond to the sum of all sample volumes taken. The pressure reduction at the sampling tap and subsequent passage of nitrogen through the liquid sample withdrawn results in the escape of the dissolved hydrogen sulfide. Subsequently, methanesulfonic acid dissolved in the organic phase (w=0.2%) is neutralized with sodium hydroxide solution. The organic liquid phase freed of acid is analyzed by means of gas chromatography, in which reactant (olefin), main product (thiol) and secondary component (tioether) can be distinguished. The results are listed in Table 1.

TABLE 1

| t/min | Conversion/% | Selectivity/% | Yield/% |
|---|---|---|---|
| 0 | 3.81 | 27.81 | 1.06 |
| 1 | 27.98 | 89.57 | 25.06 |
| 2 | 55.53 | 94.73 | 52.61 |
| 3 | 58.02 | 95.47 | 55.39 |
| 5 | 59.78 | 95.98 | 57.38 |
| 10 | 68.04 | 96.33 | 65.54 |
| 30 | 81.78 | 97.20 | 79.49 |
| 240 | 89.56 | 97.75 | 87.55 |

### Example 2

### Batchwise Stirred Vessel Experiments for Comparison of Methanesulfonic Acid with Sulfuric Acid

Performance of batchwise experiments for testing various liquid acid catalysts are carried out in a stirred autoclave (V=0.3 1) which is equipped with a sparging stirrer, baffles and a heatable jacket.

Dodecene and liquid acid are introduced before the reactor is closed. Subsequently, the reactor contents are heated to the desired reaction temperature with the stirrer motor running and the air-containing gas phase of the reactor is displaced by repeatedly injecting 10 bar of nitrogen and in each case decompressing to standard pressure. Before the hydrogen sulfide is introduced, the stirrer is switched off, so that very little hydrogen sulfide goes into solution during the three injections of H₂S (g) with subsequent decompression. Hydrogen sulfide is then finally injected into the system to the desired reaction pressure and the stirrer of the autoclave is starred immediately thereafter. During the experiment, the H₂S feed line of the reactor remains open, so that the amount of hydrogen sulfide which has reacted can be supplied subsequently into the reaction system. These experiments are evaluated both quantitatively and qualitatively by the H₂S consumption against time and gas chromatography analyses of the liquid phase.

The experiments were each carried out with 60 g of dodecene, and at a temperature of 80° C., an H₂S pressure of 10 bar and a rotation rate of the stirrer of 1600 min⁻¹. The results are listed in Table 2:

TABLE 2

| | | | | | GC analysis results | | |
|---|---|---|---|---|---|---|---|
| | | | | | [% by weight] | | |
| Experiment | Acid | m(acid) [g] | $\frac{n ( acid )}{n ( olefin )}$ | Time [min] | Dodecene | Thiol | Thioether |
| 1 | H₂SO₄ | 93.8 | 2.69 | 45 | 45.1 | 3.2 | 51.7 |
| 2 | CH₃—SO₃H | 40.0 | 1.17 | 36 | 2.5 | 91.7 | 5.8 |

The results in Table 2 show that a high conversion rate is not achieved with sulfuric acid in comparison to methanesulfonic acid in spite of a distinctly higher molar use amount. In addition, when sulfuric acid is used, a majority of the amount of thiol formed reacts further to give the thioether subsequent product, while the reaction wit methanesulfonic acid selectively affords the corresponding thiol compound.

1. A process for preparing alkyl thiols by adding hydrogen sulfide to double bonds of C₆-C₂₀ olefins at a temperature of from 20 to 150° C. and a pressure of from 1 to 40 bar, which comprises carrying out the reaction in the presence of at least one organic, liquid acid.

2. The process according to claim 1, wherein the reaction is carried out at a temperature of from 30 to 100° C.

3. The process according to claim 1, wherein C₆-C₁₈ olefins are used.

4. The process according to claim 1, wherein tertiary alkyl thiols are prepared.

5. The process according to claim 4, wherein tert-dodecyl thiol is prepared from dodecene and hydrogen sulfide.

6. The process according to claim 1, wherein the acid is selected from the group consisting of organic carboxylic acids having a total of 1 to 12 carbon atoms and alkylsulfonic acids of the general formula (X)
R—SO₃H (X)
where R is a linear or branched, saturated or unsaturated alkyl radical having from 1 to 10 carbon atoms.

7. The process according to claim 1, wherein the acid is methanesulfonic acid.

8. A method for increasing the selectivity and/or reaction rate in the addition of hydrogen sulfide to double bonds of C₆-C₂₀ olefins to prepare alkyl thiols comprising the step of adding organic, liquid acids as a catalyst.

9. The method according to claim 8, wherein the acid is methanesulfonic acid.

10. The method according to claim 8, wherein tert-dodecyl thiol is prepared by adding hydrogen sulfide to dodecene.

11. The method according to claim 9, wherein tert-dodecyl thiol is prepared by adding hydrogen sulfide to dodecene.
